(19) United States
(12) Patent Application Publication
(10) Pub. No.: US 2026/0337832 A1
(43) Pub. Date: Oct. 29, 2026

(54) PERFORMANCE STRUCTURE AND METHOD OF MANUFACTURING SAME

(71) Applicant: Thinx Inc., Neenah, WI (US)

(72) Inventor: Matt WESTERGREN-KELLY, New York, NY (US)

(73) Assignee: Thinx Inc., Neenah, WI (US)

(21) Appl. No.: 19/111,454

(22) PCT Filed: Sep. 13, 2023
(86) PCT No.: PCT/US2023/032651

§ 371 Date: Mar. 13, 2025

Related U.S. Application Data
(60) Provisional application No. 63/406,209, filed on Sep. 13, 2022.

(51) Int. Cl. D04B 1/12; D04B 1/10

(52) U.S. Cl. CPC D04B 1/12; D04B 1/108; D10B 2201/02; D10B 2201/22; D10B 2201/24; D10B 2331/04; D10B 2331/06; D10B 2331/12; D10B 2401/021; D10B 2401/022; D10B 2403/0114; D10B 2501/06

(57) ABSTRACT

A gusset performance structure having at least two surfaces, and a method of preparing same. One of the at least two surfaces is a face surface and the other of the at least two surfaces is a rear surface. The face surface faces a user and a rear surface is opposite to the face surface. The face surface is a moisture-wicking surface and the rear surface is a moisture-absorbent surface. The gusset performance structure is capable of one-way moisture transport from the face surface to the rear surface and engineered to hold a large volume of bodily fluids in a minimum amount of surface area.

### CROSS REFERENCE TO RELATED APPLICATIONS

This patent application claims the benefit of U.S. Provisional Patent Application 63/406,209 filed 13 Sep. 2022; which is incorporated by reference herein for all purposes.

### TECHNICAL FIELD

This disclosure relates to a performance structure, which includes a wicking surface and a moisture-absorbing surface, and a method of manufacturing same.

### BACKGROUND

In this disclosure, where a document, an act, and/or an item of knowledge is referred to and/or discussed, then such reference and/or discussion is not an admission that the document, the act, and/or the item of knowledge and/or any combination thereof was at a priority date, publicly available, known to a public, part of common general knowledge, and/or otherwise constitutes any prior art under any applicable statutory provisions; and/or is known to comprise relevant to any attempt to solve any problem with which this disclosure is concerned with. Further, nothing is disclaimed.

There is a desire to enable a single layer performance structure that includes both surface wicking and moisture transport to another portion of the single layer to improve moisture absorbency and comfort for a user, and that is capable of being engineered to hold a volume of a variety of bodily fluids, including, without limitation, sweat, urine, blood and menses, in a minimal surface area. However, despite various attempts by others, this type of single layer structure and functionality does not yet exist. As such, this disclosure enables such type of performance structure, including all possible combinations of any specific features described herein.

### SUMMARY

This disclosure at least partially addresses at least one of above inefficiencies. However, this disclosure can prove useful to other technical areas. Therefore, various claims recited below should not be construed as necessarily limited to addressing any of the above inefficiencies.

According to an exemplary embodiment, a device comprises at least two surfaces, wherein one of the at least two surfaces is a face surface and the other of the at least two surfaces is a rear surface, wherein the face surface is a moisture-wicking surface, wherein the rear surface is a moisture-absorbent surface, and wherein the face surface faces a user and a rear surface is opposite to the face surface.

In each of the embodiments described herein, the term yarn can refer to any material or fiber, including, for example, natural yarn, natural fibers, cellulosic fibers (seed, bast, leaf or grass), natural protein fibers (hair, fur, or secretion) synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof.

In another exemplary embodiment, the face surface comprises a hydrophobic yarn in an amount of about 29% to about 62%, and a hydrophilic yarn in an amount of at least about 5% based on a total composition of fabric weight. In certain embodiments, the face surface is predominantly hydrophobic yarn. The amount of the hydrophobic yarn situated in the face surface can be from about 51% to about 95%. In certain embodiments, the face surface comprises about 40% hydrophilic yarn.

In another exemplary embodiment, the rear surface comprises a hydrophilic yarn in an amount of from about 38% to about 71% based on a total composition of fabric weight. In certain embodiments, the rear surface is predominantly hydrophilic yarn. The amount of the hydrophilic yarn situated in the rear surface can be from about 49% to 95%.

In another exemplary embodiment, the rear surface comprises fibers in an amount that is greater than an amount of fibers in the face surface.

In another exemplary embodiment, the face surface comprises a moisture-wicking material selected from natural yarn, natural fibers, cellulosic fibers (seed, bast, leaf or grass), natural protein fibers (hair, fur, or secretion) synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof.

In another exemplary embodiment, the rear surface comprises a moisture-absorbing material, which can be selected from, natural yarn, natural fibers, cellulosic fibers (seed, bast, leaf or grass), natural protein fibers (hair, fur, or secretion) synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof. In a preferred embodiment, the rear surface comprises Novel cellulosic staple. In another preferred embodiment, the rear surface comprises Lyocell available as Dtex 40f24 (1.66 dpf), Dtex 80f48 (1.66 dpf), Dtex 120f73 (1.64 dpf), Dtex 160f93 (1.72 dpf).

In another exemplary embodiment, the at least two surfaces can form a single layer, where a first surface of the single layer is the face surface and a second surface of the single layer opposite to the first layer is a rear surface.

In another exemplary embodiment, the at least two surfaces can form two layers, wherein the face surface is a face layer and the rear surface is a back layer.

In another exemplary embodiment, the device can further comprise a barrier layer positioned on the rear surface opposite to the face surface.

In another exemplary embodiment, the face surface may comprise fibers in an amount of 1.0-3.0 denier per filament.

In another exemplary embodiment, the face surface may comprise microfibers or fibers in an amount of 0.5-1.1 denier per filament. In certain embodiments, the face surface may comprise fibers in an amount of 1.04 denier per filament.

In another exemplary embodiment, the face and rear surfaces are formed concurrently in a pattern. In certain embodiments, the pattern is a linear knit pattern, a geometric dobby knit pattern, a jacquard knit pattern, a rectangular knit pattern, a triangle knit pattern, a square knit pattern, a pentagon knit pattern, a hexagon knit pattern, a heptagon knit pattern, an octagon knit pattern, a nonagon knit pattern, a decagon knit pattern, or any other polygonal knit pattern.

In another exemplary embodiment, each of the front and rear surfaces has a single or double pique structure. In certain embodiments the invention could be constructed using a plain weave structure, a basket weave structure, a twill weave structure, a satin weave structure, a dobby weave structure, a jacquard weave structure, a crepe weave structure, a double-cloth weave structure, a pile or loop weave structure, a single or double jersey knit structure, an interlock knit structure, a jacquard knit structure, a tricot knit structure, a raschel knit structure, a lace knit structure, an intarsia knit structure, a terry loop knit structure, a velour knit structure a fleece knit structure or any combination of the above structures.

In another exemplary embodiment, the face surface comprises a pre-colored yarn.

In another exemplary embodiment, the rear surface comprises an undyed yarn.

In another exemplary embodiment, a water absorption time of the face surface is from about 3 seconds to 60 seconds.

In another exemplary embodiment, a water absorption time of the rear surface is about 10 seconds to about 179 seconds.

In another exemplary embodiment, a water absorption capacity of each of the face and rear surfaces is about 200% to about 400%.

Another exemplary embodiment is directed to a gusset performance structure comprising the device described above.

In another exemplary embodiment, a weight of the performance structure is about 200 gsm to about 450 gsm.

In another exemplary embodiment, a width of the performance structure is about 0.5 mm to about 10 mm.

In another exemplary embodiment, a water absorption capacity of the performance structure is about 5 ml to about 150 ml.

In another exemplary embodiment, a plurality of performance structures may be layered, such as, for example from five to fifteen layers, to provide a water absorption capacity of about 750 ml.

Another exemplary embodiment is directed to a device comprising a gusset performance structure and at least one additional material layer.

In another exemplary embodiment, a material of the at least one material layers is selected from fabric, natural yarn, natural fibers, cellulosic fibers (seed, bast, leaf or grass), natural protein fibers (hair, fur, or secretion) synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof.

In another exemplary embodiment, the performance structure is bonded or adhered to the at least one material layer.

In another exemplary embodiment, the performance structure and the material layer are bonded or adhered together via a stitching, a knitting, an adhesive layer or a bonding tape.

Another exemplary embodiment is directed to a method comprising: causing a device to comprise at least two surfaces, wherein one of the at least two surfaces is a face surface and the other of the at least two surfaces is a rear surface, causing the face surface to be positioned in a direction facing a user, causing the rear surface to be positioned in a direction opposite to the face surface, causing the face surface to comprise a moisture-wicking material, and causing the rear surface to comprise a moisture-absorbent material.

In another exemplary embodiment, the method can further comprise forming the face and rear surfaces concurrently during manufacture.

In another exemplary embodiment, the method can further comprise forming a single or double pique pattern in each of the front and rear surfaces.

In another exemplary embodiment, the method can further comprise causing the face surface to comprise a pre-colored yarn.

In another exemplary embodiment, the method can further comprise causing the rear surface to comprise an undyed yarn.

This disclosure is embodied in various forms illustrated in a set of accompanying illustrative drawings. Note that variations are contemplated as being a part of this disclosure, limited only by a scope of various claims recited below.

### DESCRIPTION OF DRAWINGS

The set of accompanying illustrative drawings shows various example embodiments of this disclosure. Such drawings are not to be construed as necessarily limiting this disclosure. Like numbers and/or similar numbering scheme can refer to like and/or similar elements throughout.

FIG. 1 is an illustration of a performance structure according to an exemplary embodiment.

FIG. 2 is an illustration of a face surface of the performance structure of FIG. 1.

### DETAILED DESCRIPTION OF PREFERRED EMBODIMENTS

This disclosure is now described more fully with reference to the set of accompanying illustrative drawings, in which example embodiments of this disclosure are shown. This disclosure can be embodied in many different forms and should not be construed as necessarily being limited to the example embodiments disclosed herein. Rather, the example embodiments are provided so that this disclosure is thorough and complete, and fully conveys various concepts of this disclosure to those skilled in a relevant art.

Features described with respect to certain example embodiments can be combined and sub-combined in and/or with various other example embodiments. Also, different aspects and/or elements of example embodiments, as disclosed herein, can be combined and sub-combined in a similar manner as well. Further, some example embodiments, whether individually and/or collectively, can be components of a larger system, wherein other procedures can take precedence over and/or otherwise modify their application. Additionally, a number of steps can be required before, after, and/or concurrently with example embodiments, as disclosed herein. Note that any and/or all methods and/or processes, at least as disclosed herein, can be at least partially performed via at least one entity in any manner.

Various terminology used herein can imply direct or indirect, full or partial, temporary or permanent, action or inaction. For example, when an element is referred to as being “on,” “connected” or “coupled” to another element, then the element can be directly on, connected or coupled to the other element and/or intervening elements can be present, including indirect and/or direct variants. In contrast, when an element is referred to as being “directly connected” or “directly coupled” to another element, there are no intervening elements present.

Although the terms first, second, etc., can be used herein to describe various elements, components, regions, layers and/or sections, these elements, components, regions, layers and/or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from various teachings of this disclosure.

Various terminology used herein is for describing particular example embodiments and is not intended to necessarily limit this disclosure. As used herein, various singular forms “a,” “an” and “the” are intended to include various plural forms as well, unless a context clearly indicates otherwise. Various terms “comprises,” “includes” and/or “comprising,” “including” when used in this specification, specify a presence of stated features, integers, steps, operations, elements, and/or components, but do not preclude the presence and/or addition of one or more other features, integers, steps, operations, elements, components, and/or groups thereof.

As used herein, a term “or” is intended to mean an inclusive “or” rather than an exclusive “or.” That is, unless specified otherwise, or clear from context, “X employs A or B” is intended to mean any of a set of natural inclusive permutations. That is, if X employs A; X employs B; or X employs both A and B, then “X employs A or B” is satisfied under any of the foregoing instances.

As used herein, the term “stitching” includes any stitching bonding pattern that can connect two or more layers, without being limited thereto. Any such stitching pattern may be seamless, but is not limited thereto.

As used herein, the term “knitting” includes any knitting fabric formation pattern that can connect two or more layers, without being limited thereto. Any such knitting pattern may be seamless, but is not limited thereto.

As used here, the term “performance structure” includes a material that is used to form into a garment to help shape and reinforce key points, add breadth, reduce stress, and the like, in clothing. A performance structure can be included at any position in a garment, including but not limited to the shoulders, underarms, hemlines, crotches, and the like. The shape of the performance structure is not particularly limited, and can include any suitable shape, including but not limited to a triangular, rhomboidal, diamond shape. The performance structure can be used throughout the garment to provide performance features as described herein.

As used here, the term “gusset performance structure” includes a panel that is inserted into a garment to help shape and reinforce key points, add breadth, reduce stress, and the like, in clothing. A gusset performance structure can be included at any position in a garment, including but not limited to the shoulders, underarms, hemlines, crotches, and the like. The shape of the gusset performance structure is not particularly limited, and can include any suitable shape, including but not limited to a triangular, rhomboidal, diamond shape.

Example embodiments of this disclosure are described herein with reference to illustrations of idealized embodiments (and intermediate structures) of this disclosure. As such, variations from various illustrated shapes as a result, for example, of manufacturing techniques and/or tolerances, are to comprise expected variations. Thus, various example embodiments of this disclosure should not be construed as necessarily limited to various particular shapes of regions illustrated herein, but are to include deviations in shapes that result, for example, from manufacturing.

Any and/or all elements, as disclosed herein, can be and/or include, any type of fabric, without any limitation as long as they have the required properties as described herein. Any and/or all elements, as disclosed herein, can be and/or include, whether partially and/or fully, a solid, including a metal, a mineral, an amorphous material, a ceramic, a glass ceramic, an organic solid, such as wood and/or a polymer, such as rubber, a composite material, a semiconductor, a nanomaterial, a biomaterial and/or any combinations thereof. Any and/or all elements, as disclosed herein, can be and/or include, whether partially and/or fully, a coating, including an informational coating, such as ink, an adhesive coating, a melt-adhesive coating, such as vacuum seal and/or heat seal, a release coating, such as tape liner, a low surface energy coating, an optical coating, such as, for example, tint, color, hue, saturation, tone, shade, transparency, translucency, opaqueness, luminescence, reflection, phosphorescence, anti-reflection and/or holography, a photo-sensitive coating, an electronic and/or thermal property coating, such as for passivity, insulation, resistance or conduction, a magnetic coating, a water-resistant and/or waterproof coating, a scent coating and/or any combinations thereof. Any and/or all elements, as disclosed herein, can be rigid, flexible, and/or any other combinations thereof. Any and/or all elements, as disclosed herein, can be identical and/or different from each other in material, shape, size, color and/or any measurable dimension, such as length, width, height, depth, area, orientation, perimeter, volume, breadth, density, temperature, resistance, and so forth.

Unless otherwise defined, all terms (including technical and scientific terms) used herein have the same meaning as commonly understood by one of ordinary skill in an art to which this disclosure belongs. Various terms, such as those defined in commonly used dictionaries, should be interpreted as having a meaning that is consistent with a meaning in a context of a relevant art and should not be interpreted in an idealized and/or overly formal sense unless expressly so defined herein.

Furthermore, relative terms such as “below,” “lower,” “above,” and “upper” can be used herein to describe one element's relationship to another element as illustrated in the set of accompanying illustrative drawings. Such relative terms are intended to encompass different orientations of illustrated technologies in addition to an orientation depicted in the set of accompanying illustrative drawings. For example, if a device in the set of accompanying illustrative drawings were turned over, then various elements described as being on a “lower” side of other elements would then be oriented on “upper” sides of other elements. Similarly, if a device in one of illustrative figures were turned over, then various elements described as “below” or “beneath” other elements would then be oriented “above” other elements. Therefore, various example terms “below” and “lower” can encompass both an orientation of above and below.

As used herein, a term “about” and/or “substantially” refers to a +/−10% variation from a nominal value/term. Such variation is always included in any given value/term provided herein, whether or not such variation is specifically referred thereto.

If any disclosures are incorporated herein by reference and such disclosures conflict in part and/or in whole with this disclosure, then to an extent of a conflict, if any, and/or a broader disclosure, and/or broader definition of terms, this disclosure controls. If such disclosures conflict in part and/or in whole with one another, then to an extent of a conflict, if any, a later-dated disclosure controls.

This application is directed to a single textile performance structure, comprising a smooth, yet discontinuous, face surface optimized for surface wicking and a moisture transport to a highly absorbent rear surface, that is capable of being engineered to hold a large volume of a variety of bodily fluids within a minimum surface area, wherein the device is capable of moisture transport from the face side to the rear side. In certain embodiments, the performance structure is a gusset performance structure.

The face surface, which has a wicking property, can be constructed from hydrophobic synthetic yarns having low denier and high filament count. Fibers in the range of 0.5-1.5 denier per filament are preferred. In certain embodiments, a Ne16 (370 dtex) cotton yarn is used. A multi-lobal or multi-channel cross section can be used to maximize capillary forces and overall wicking ability.

The rear surface, which has an absorbing property, can be constructed from hydrophilic staple or continuous filament yarns. These hydrophilic yarns can have a higher denier than the yarns used in the face surface. If continuous filament yarns are uses, an oval, lobed or striated cross section can be used to enhance capillary forces and overall wicking ability. In certain embodiments, the rear surface can comprise float and/or loops of a predetermined length, direction, and pattern. The float/loop density is determined by the overall absorbent properties of the hydrophilic raw material and desired product liquid volume holding requirement.

In certain embodiments, a flat, double pique construction is provided. Such construction provides a beneficial performance for the textile.

A process of constructing the face and rear surfaces can include a transfer of yarns from the face surface to the rear surface, and vice versa, resulting in a defined inverted pattern of a specific repeat, shape, and size. Upon selective coloration, the face surface will be of a dark shade, whereas the rear surface yarns will be undyed and clearly visible in the technical face. The resulting pattern can be used as a visual guide for the presence of fluid within the textile structure.

The gusset performance structure can be included in a fabric. The resultant fabric can be finished in capable of offering specific anti-bacterial, anti-odor, anti-static, moisture management, stain repellent, fragrance, & general easy-care properties targeted toward the product end use and selected with minimal impact to the inherent physical properties of the chosen raw materials. Manufacture of the described textile may be either knitted or woven, fully conforming to the performance and durability requirements of the end-product.

The performance structure or a material including the performance structure can also work in conjunction with additional textile layers in end-product uses that demand the absorption, movement and retention of fluid, such other layers maybe films, laminates, coatings or other barrier technologies if desired.

The end-products for which a performance structure or a material including the performance structure can also work in conjunction with additional textile layers can include, for example, activewear, athletic wear, casualwear, swimwear, shapewear, uniforms and hospitality wear, patients and healthcare, formal and occasion wear, trousers, bottoms, leggings, tights, shorts, boxers, trunks, briefs, bras, and underarm pads.

The absorption of the gusset performance structure can be maximized by selecting moisture absorbing materials capable of retaining the greatest volume of moisture absorbed in the smallest surface area.

The performance structure and its constituent materials may be selected to provide a balance of absorption via physical means, such as capillary forces between the fibers and yarns, and through hydrogen bonding within the material structure itself to minimize any potential for moisture to be transferred to the wearer in use.

The high-wicking material of the face surface can include a low dpf continuous filament with multi-lobal cross section, which promotes wicking after initial surface wetting. The face materials may also be any from the range of hydrophilic materials listed previously, that has been treated with a chemical agent, or process to be rendered hydrophobic The face surface can include a repeating surface pattern in contrasting color and absorbency capabilities, with the pattern visible to the naked eye. The face surface can also be dyed in a preferred color, such as black, greige, optic white, and the like, but are not limited thereto. A regular series of tuck and float stitches can be used to invert face and rear materials.

The comfort and feel of the performance structure is optimized by minimizing thermal capacity, reducing layers within the structure, minimizing the heigh of any surface texture, creating a single layer textile from low thermally retentive materials, increasing breathability, and using a film or carrier textile with enhanced air/vapor permeability.

In other exemplary embodiments, the odor control can be optimized in the performance structure, different chemistries can be incorporated in the performance structure according to the intended end use, a chemical finish can be engineered for a specified range of VOC, chemical finishes can be minimized to enhance safety and comfort, the performance structure can be optimized for all bodily fluids.

Moisture management can be optimized by using synthetic/cellulosic blends for the performance structure. Preferably, the synthetic/cellulosic blends can include cellulosic (natural & regenerated) and recycled synthetic components. A raw material differential is required to create the desired one-way moisture management effect in the performance structure; preferably, the performance structure has a high differential in yarn linear density, with a lower density of yarns in the face surface compared to the rear surface. In a preferred embodiment, the face and rear surface may comprise a combed ring-spun yarn. In an alternate embodiment, the rear surfaces comprise continuous filaments.

In an exemplary embodiment, the face surface comprises recycled polyester fibers having about 1 dpf. The face surface can be flat or have a channeled cross-section. The face surface may have a cationic dyeable polyester (CDP) or hydrophilic finish. The yarns of the face surface may be doped or jet-dyed a desired color. The yarn count of the rear surface is dependent on the type of fiber blend, but preferably, a coarser texture is preferred to optimize moisture management capabilities.

In certain embodiments, the fibers can be 150 dtex, 144 filament (1.04 dpf) for the face surface, and 370 dtex, Ne16 filament for the rear surface.

In certain embodiments, the yarns can be doped or dyed any color, such as, for example, red, orange, yellow, green, blue, indigo, violet, brown, black, or any other color or combination thereof.

The yarns for the face surface can be white, optic white, or undyed. The face surface can be a two-face, or any other structure constructed via any technique that enables preferential selection and placement of yarns. In certain embodiments the invention could be constructed using a plain weave structure, a basket weave structure, a twill weave structure, a satin weave structure, a dobby weave structure, a single or double pique weave structure, a jacquard weave structure, a crepe weave structure, a double-cloth weave structure, a pile or loop weave structure, a single or double jersey knit structure, an interlock knit structure, a jacquard knit structure, a tricot knit structure, a raschel knit structure, a lace knit structure, an intarsia knit structure, a single or double knit structure, a terry loop knit structure, a velour knit structure a fleece knit structure or any combination of the above structures.

According to an exemplary embodiment, a device comprises at least two surfaces, wherein one of the at least two surfaces is a face surface and the other of the at least two surfaces is a rear surface, wherein the face surface is a moisture-wicking surface, wherein the rear surface is a moisture-absorbent surface, and wherein the face surface faces a user and a rear surface is opposite to the face surface.

In another exemplary embodiment, the face surface comprises a hydrophobic yarn in an amount of about 29% to about 62%, about 29% to about 39%, about 29% to about 39%, about 35% to about 62%, about 40% to about 60%, about 50% to about 60%, about 85% to about 90%, and the like, and a hydrophilic yarn in an amount of at least about 5% to about 49%, about 7% to about 40%, about 7% to about 30%. About 8% to about 25%, about 9% to about 20%, about 10% to about 15% based on a total composition of the face surface, and can include any suitable values within these ranges. For example, the percentage of hydrophobic yarn can be in an amount of about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, or any range of percentages or percentages that include one or more of the foregoing percentages, or any percentage in between the foregoing percentages. For example, the percentage of hydrophilic yarn can be in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, or any range of percentages or percentages that include one or more of the foregoing percentages, or any percentage in between the foregoing percentages, or any range of percentages or percentages that include one or more of the foregoing percentages, or any percentage in between the foregoing percentages.

In another exemplary embodiment, the rear surface comprises a hydrophilic yarn in an amount of from about 38% to about 71%, about 39% to about 71%, about 40% to about 71%, about 41% to about 71%, about 51% to about 71%, about 61% to about 71%, about 40% to about 60%, about 45% to about 55%, and the like, based on a total composition of the rear surface, and can include any suitable value within these ranges. For example, the percentage of hydrophilic yarn can be in an amount of about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or any range of percentages or percentages that include one or more of the foregoing percentages, or any percentage in between the foregoing percentages. A preferred amount of the hydrophilic yarn can be about 51% to about 71% and about 61% to about 71%.

In another exemplary embodiment, the rear surface comprises microfibers in an amount that is greater than the amount of microfibers in the face surface.

In another exemplary embodiment, the face surface comprises a moisture-wicking material selected from natural yarn, natural fibers, cellulosic fibers (seed, bast, leaf or grass), natural protein fibers (hair, fur, or secretion), synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof.

In another exemplary embodiment, the rear surface comprises a material, which can be selected from natural yarn, natural fibers, cellulosic fibers (seed, bast, leaf or grass), natural protein fibers (hair, fur, or secretion) synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof.

In another exemplary embodiment, the at least two surfaces can form a single layer, where a first surface of the single layer is the face surface and a second surface of the single layer opposite to the first layer is a rear surface.

In another exemplary embodiment, the at least two surfaces can form two layers, wherein the face surface is a face layer and the rear surface is a back layer.

In another exemplary embodiment, the device can further comprise a barrier layer positioned on the rear surface opposite to the face surface.

In another exemplary embodiment, the face surface comprises fibers or microfibers in an amount of about 0.5 denier to about 1.5 denier, about 0.55 denier to about 1.3 denier, about 0.6 denier to about 1.25 denier, about 0.65 denier to about 1.25 denier, about 0.7 denier to about 1.1 denier per filament, and any suitable value within these ranges. For example, microfibers may be in an amount of about 0.50 denier, about 0.55 denier, about 0.60 denier, about 0.65 denier, about 0.70 denier, about 0.75 denier, about 0.8 denier, about 0.85 denier, about 0.9 denier, about 0.95 denier, about 0.96 denier, about 0.97 denier, about 0.98 denier, about 0.99 denier, about 1.0 denier, about 1.05 denier, about 1.1 denier, about 1.15 denier, about 1.20 denier, about 1.25 denier, about 1.30 denier, about 1.35 denier, about 1.40 denier, about 1.45 denier, about 1.50 denier, or any range of deniers that include one or more of the foregoing amounts, or any amount in between the foregoing deniers.

In another exemplary embodiment, the face and rear surfaces are formed concurrently in a circular knit pattern.

In another exemplary embodiment, each of the front and rear surfaces has a single or double pique pattern.

In another exemplary embodiment, the face surface comprises a pre-colored yarn.

In another exemplary embodiment, the rear surface comprises an undyed yarn.

In another exemplary embodiment, a water absorption time of the face surface is about 3 seconds to about 30 seconds, about 3.5 seconds to about 15 seconds, about 4 seconds to about 10 seconds, about 4.5 seconds to about 5 seconds, and the like, including any suitable value within these ranges. For example, the water absorption time may be about 3 seconds, about 3.5 seconds, about 4 seconds, about 4.5 seconds, about 5 seconds, about 5.5 seconds, about 6 seconds, about 6.5 seconds, about 7 seconds, about 7.5 seconds, about 8 seconds, about 8.5 seconds, about 9 seconds, about 9.5 seconds, about 10 seconds, about 10.5 seconds, about 11 seconds, about 11.5 seconds, about 12 seconds, about 12.5 seconds, about 13 seconds, about 13.5 seconds, about 14 seconds, about 14.5 seconds, about 15 seconds, about 15.5, seconds, about 16 seconds, about 16.5 seconds, about 17 seconds, about 17.5 seconds, about 18 seconds, about 18.5 seconds, about 19 seconds, about 19.5 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, or any range of time or amount of time that include one or more of the foregoing amounts, or any amount in between the foregoing times.

In another exemplary embodiment, a water absorption time of the rear surface is about 10 seconds to about 200 seconds, about 15 seconds to about 150 seconds, about 20 seconds to about 100 seconds, about 25 seconds to about 75 seconds, about 30 seconds to about 70 seconds, and the like, including any suitable value within these ranges. For example, the water absorption time may be about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 60 seconds, about 65 seconds, about 70 seconds, about 75 seconds, about 80 seconds, about 85 seconds, about 90 seconds, about 95 seconds, about 100 seconds, about 110 seconds, about 120 seconds, about 130 seconds, about 140 seconds, about 150 seconds, about 160 seconds, about 170 seconds, about 180 seconds, about 190 seconds, about 200 seconds, or any range of time or amount of time that include one or more of the foregoing amounts, or any amount in between the foregoing times.

In another exemplary embodiment, a water absorption capacity of each of the face and rear surfaces is about 200% to about 350%, about 210% to about 340%, about 220% to about 330%, about 230% to about 320%, about 240% to about 310%, about 250% to about 300%, and the like, including any suitable value within these ranges. For example, the water absorption capacity of each of the face and rear surfaces may be about 200%, about 210%, about 220%, about 230%, about 240%, about 250%, about 260%, about 270%, about 280%, about 290%, about 300%, about 310%, about 320%, about 330%, about 340%, about 350%, or any percentage or range of percentages that include one or more of the foregoing percentages, or any percentage in between the foregoing percentages.

Another exemplary embodiment is directed to a gusset performance structure comprising the device described above.

In another exemplary embodiment, a weight of the gusset performance structure is about 200 g to about 300 g, about 205 g to about 295 g, about 210 g to about 290 g, about 215 g to about 285 g, about 220 g to about 280 g, about 225 g to about 275 g, about 230 g to about 270 g, about 235 g to about 265 g, and the like, including any suitable value within these ranges. For example, the weight of the gusset performance structure is about 200 g, about 205 g, about 210 g, about 215 g, about 220 g, about 225 g, about 230 g, about 235 g, about 240 g, about 245 g, about 250 g, about 255 g, about 260 g, about 265 g, about 270 g, about 275 g, about 280 g, about 285 g, about 290 g, about 295 g, about 300 g, or any range of weights or weights including one or more of the foregoing or any weight in between the foregoing.

In another exemplary embodiment, a depth or thickness of the gusset performance structure is about 4 mm to about 10 mm, about 5 mm to about 9 mm, about 6 mm to about 8 mm, about 7 mm to about 9 mm, about 8 mm to about 9 mm, and the like, including any suitable value within these ranges. For example, the depth of the gusset performance structure may be about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5.0 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, about 6.1 mm, about 6.2 mm, about 6.3 mm, about 6.4 mm, about 6.5 mm, about 6.6 mm, about 6.7 mm, about 6.8 mm, about 6.9 mm, about 7.0 mm, about 7.1 mm, about 7.2 mm, about 7.3 mm, about 7.4 mm, about 7.5 mm, about 7.6 mm, about 7.7 mm, about 7.8 mm, about 7.9 mm, about 7.0 mm, about 8.1 mm, about 8.2 mm, about 8.3 mm, about 8.4 mm, about 8.5 mm, about 8.6 mm, about 8.7 mm, about 8.8 mm, about 8.9 mm, about 9.0 mm, about 9.1 mm, about 9.2 mm, about 9.3 mm, about 9.4 mm, about 9.5 mm, about 9.6 mm, about 9.7 mm, about 9.8 mm, about 9.9 mm, about 10.0 mm, about 10.5 mm, about 11 mm, about 11.5 mm, about 12 mm, about 12.5 mm, about 13 mm, about 13.5 mm, about 14 mm, about 14.5 mm, about 15 mm, or any depth or range of depths or thicknesses that include one or more of the foregoing depths or thicknesses, or any depth in between the foregoing depths or thicknesses.

In another exemplary embodiment, a water absorption capacity of the gusset performance structure is about 250 ml to about 1000 ml, about 275 ml to about 950 ml, about 300 ml to about 900 ml, about 325 ml to about 850 ml, about 350 ml to about 800 ml, about 375 ml to about 750 ml, about 400 ml to about 700 ml, about 425 ml to about 650 ml, about 450 ml to about 600 ml, and the like, including any suitable value within these ranges. For example, the water absorption capacity of the gusset performance structure may be about 250 ml, about 275 ml, about 300 ml, about 325 ml, about 350 ml, about 375 ml, about 400 ml, about 425 ml, about 450 ml, about 475 ml, about 500 ml, about 525 ml, about 550 ml, about 575 ml, about 600 ml, about 625 ml, about 650 ml, about 675 ml, about 700 ml, about 725 ml, about 750 ml, about 775 ml, about 800 ml, about 825 ml, about 850 ml, about 875 ml, about 900 ml, about 925 ml, about 950 ml, about 975 ml, about 1000 ml, or any water absorbance or range of water absorbance that include one or more of the foregoing amounts, or any amount in between the foregoing water absorbance amounts.

In another exemplary embodiment, the area of the gusset performance structure is between 35 square inches to 120 square inches.

Another exemplary embodiment is directed to a device comprising the gusset performance structure and at least one material layer.

In another exemplary embodiment, a material of the at least one material layers is selected from fabrics, natural yarn, natural fibers, cellulosic fibers (seed, bast, leaf or grass), natural protein fibers (hair, fur, or secretion) synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof.

In another exemplary embodiment, the gusset performance structure is bonded or adhered to the at least one material layer.

In another exemplary embodiment, the gusset performance structure and the material layer are bonded or adhered together via a stitching, a knitting, an adhesive layer or a bonding tape.

Another exemplary embodiment is directed to a method comprising: causing a device to comprise at least two surfaces, wherein one of the at least two surfaces is a face surface and the other of the at least two surfaces is a rear surface, causing the face surface to be positioned in a direction facing a user, causing the rear surface to be positioned in a direction opposite to the face surface, causing the face surface to comprise a moisture-wicking material, and causing the rear surface to comprise a moisture-absorbent material.

In another exemplary embodiment, the method can further comprise forming the face and rear surfaces concurrently in a circular knit pattern.

In another exemplary embodiment, the method can further comprise forming a single or double pique pattern in each of the front and rear surfaces.

In another exemplary embodiment, the method can further comprise causing the face surface to comprise a pre-colored yarn.

In another exemplary embodiment, the method can further comprise causing the rear surface to comprise an undyed yarn.

The device according to any one of the prior exemplary embodiments, wherein the gusset performance structure can be combined with one or more material layers independently selected from fabric, natural yarn, natural fibers, cellulosic fibers (seed, bast, leaf or grass), natural protein fibers (hair, fur, or secretion) synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof. However, material of any one or more of the material layers is not limited to comprising the above-described materials, and any suitable material may be incorporated into the garment.

The thickness of each of the material layers may be from about 0.5 mm to about 10 mm, preferably from about 0.6 mm to 9 mm, from about 0.7 mm to about 8 mm, from about 0.8 mm to about 7 mm, from about 0.9 mm to about 6 mm, from about 1 mm to about 5 mm, from about 1.5 mm to about 4.5 mm, from about 2 mm to about 4 mm, from about 3 mm to about 3.5 mm, and more preferably, about 1 mm, about 1.25 mm, about 1.5 mm, about 1.75 mm, about 2.0 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, and any other suitable thickness within these ranges. For example, the thickness of each of the material layers may be about 0.5 mm, about 0.55, about 0.6 mm, about 0.65, about 0.7 mm, about 0.75, about 0.8 mm, about 0.85, about 0.9 mm, about 0.95, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, about 4.6 mm, about 4.7 mm, about 4.8 mm, about 4.9 mm, about 5.0 mm, about 5.1 mm, about 5.2 mm, about 5.3 mm, about 5.4 mm, about 5.5, about 5.6, about 5.7, about 5.8, about 5.9, about 6.0, about 6.1 mm, about 6.2 mm, about 6.3 mm, about 6.4 mm, about 6.5 mm, about 6.6 mm, about 6.7 mm, about 6.8 mm, about 6.9 mm, about 7.0 mm, about 7.1 mm, about 7.2 mm, about 7.3 mm, about 7.4 mm, about 7.5, about 7.6, about 7.7, about 7.8, about 7.9, about 8.0, about 8.1 mm, about 8.2 mm, about 8.3 mm, about 8.4 mm, about 8.5 mm, about 8.6 mm, about 8.7 mm, about 8.8 mm, about 8.9 mm, about 9.0 mm, about 9.1 mm, about 9.2 mm, about 9.3 mm, about 9.4 mm, about 9.5, about 9.6, about 9.7, about 9.8, about 9.9, about 10.0 mm, or any thickness or range of thicknesses that include one or more of the foregoing thicknesses, or any thickness in between the foregoing thicknesses.

The device according to any one of the prior exemplary embodiments, wherein the gusset performance structure may be incorporated into any such material layer.

The device according to any one of the prior exemplary embodiments, wherein the gusset performance structure may be bonded or adhered to any such material layer via a stitching, a knitting, an adhesive layer, bonding tape or any other attachment methods.

The device according to any one of the prior exemplary embodiments, wherein the moisture-wicking materials is selected from natural yarn, natural fibers, cellulosic fibers (seed, bast, leaf or grass), natural protein fibers (hair, fur, or secretion) synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof. The moisture-wicking material may be any material that allows transport of moisture, water and/or liquids. A layer comprising the moisture-wicking material may be a water/moisture absorbable layer, such as a polymer blend, including but not limited to a polyester/nylon blend (e.g., 80% polyester, 20% nylon). In various exemplary embodiments, the moisture absorbable layer may include a combination of materials such as, but not limited to, 100% polyester, 96% cotton/4% spandex, 80% polyester/20% nylon, Bramante®/cotton blend, cotton flock, natural fibers, wool, and the like. In various exemplary embodiments, the moisture-wicking material may be a combination of materials selected from fabric, natural yarn, synthetic yarn, wool, polypropylene, nylon, polyester or combinations thereof. Various, non-limiting, exemplary embodiments of the moisture-wicking layer are listed in Table 1:

TABLE 1
Composition

88 Polyester/12 Lyocell
45 Polyester/8 Elastane/47
Cotton
80 Polyester/20 Elastane
27 Cotton/73 Polyester
68 Supima Cotton/26
Polyester/6 Elastane
71 Modal/21 Polyester/8
Elastane
68 Lyocell/25 Polyester/7
Elastane
90 Polyester/10 Elastane
96 Micro Modal Air Jet/4
Elastane
48 Micromodal Compact/41
Polyamide/11 Elastane
34 Supima Cotton/34
Micromodal Air/32 Elastane
43 Viscose/39 Micromodal
Compact/18 Elastane
39 Cotton/22 Seacell/34
Modal/5 Spandex
82.7 Seacell Modal/12.3
Polyester/5 Spandex
95Ecovero Viscose/5
Spandex
56 Modal/37 Refibra/7
Spandex
89 Repreve Recycled
Polyester/11 Recycled
Spandex

However, the moisture-wicking material is not limited thereto, and any suitable moisture-wicking material may be incorporated into the garment.

The device according to any one of the prior exemplary embodiments, wherein a maximum absorption capacity of the fabric/device (gusset performance structure) is in a range of from about 5 ml to about 750 ml, preferably from about 55 ml to about 250 ml, from about 60 ml to about 200 ml, from about 65 ml to about 150 ml, and more preferably, about 50 ml, about 55 ml, about 60 ml, about 65 ml, about 70 ml, about 75 ml, about 80 ml, about 85 ml, about 90 ml, about 95 ml, about 100 ml, about 105 ml, about 110 ml, about 115 ml, about 120 ml, about 125 ml, about 130 ml, about 135 ml, about 140 ml, about 145 ml, about 150 ml, about 155 ml, about 160 ml, about 165 ml, about 170 ml, about 175 ml, about 180 ml, about 185 ml, about 190 ml, about 195 ml, about 200 ml, about 205 ml, about 210 ml, about 215 ml, about 220 ml, about 225 ml, about 230 ml, about 235 ml, about 240 ml, about 245 ml, about 250 ml, about 255 ml, about 260 ml, about 265 ml, about 270 ml, about 275 ml, about 280 ml, about 285 ml, about 290 ml, about 295 ml, about 300 ml, and/or any other suitable volume within these ranges, or any volume or range of volumes that include one or more of the foregoing volumes, or any volume in between the foregoing volumes

In some embodiments, various functions or acts can take place at a given location and/or in connection with the operation of one or more apparatuses or systems. In some embodiments, a portion of a given function or act can be performed at a first device or location, and a remainder of the function or act can be performed at one or more additional devices or locations.

The corresponding structures, materials, acts, and equivalents of all means or step plus function elements in the claims below are intended to include any structure, material, or act for performing the function in combination with other claimed elements as specifically claimed. The embodiments were chosen and described in order to concisely explain the principles of the disclosure and the practical application, and to enable others of ordinary skill in the art to understand the disclosure for various embodiments with various modifications as are suited to the particular use contemplated.

Various exemplary embodiments of this application are illustrated in the accompanying drawings.

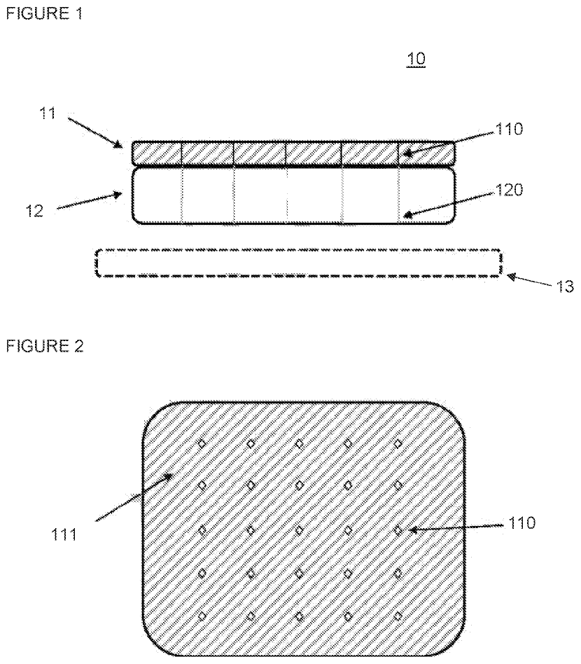

FIG. 1 is an illustration of a gusset performance structure 10 according to an exemplary embodiment. As illustrated in FIG. 1, the gusset performance structure includes a hydrophobic wicking face surface 11 and a hydrophilic moisture-absorbing rear surface 12. The face surface 11 includes undyed hydrophilic back yarn 110. The rear surface includes a space where the back yarn has been transferred to the face of the structure. In certain embodiments the rear stitch is transferred from the back of the structure to the face, and back again to the rear, forming a single stiches length of hydrophilic yarn in a repeatable pattern on the face. The gusset performance structure 10 can include an optional barrier layer 13 positioned on the rear surface. In an exemplary embodiment, the face surface 11 and rear surface 12 are formed as different layers, as illustrated in FIG. 1. However, this invention is not limited thereto, and the face surface 11 and rear surface 12 may be included as a single layer.

FIG. 2 is a top view of the face surface of the gusset performance structure of FIG. 1. As illustrated in FIG. 2, the face surface includes a combination of dyed hydrophobic yarn 111 in addition to the undyed hydrophilic yarn 110, which is positioned within the face surface. As illustrated in FIG. 2, the undyed yarn 110 forms a regular pattern extending the full width and length of the textile used to form the face surface 11. Whereas the rear of the structure now has a hole where the stitch has been transferred to the face and back again preempt

Any diagrams, images and drawings depicted herein are illustrative. There can be many variations to the diagram or the steps (or operations) described therein without departing from the spirit of the disclosure. For instance, the steps can be performed in a differing order or steps can be added, deleted or modified. All of these variations are considered a part of the disclosure. It will be understood that those skilled in the art, both now and in the future, can make various improvements and enhancements which fall within the scope of the claims which follow.

The description of this disclosure has been presented for purposes of illustration and description, but is not intended to comprise fully exhaustive and/or limited to the disclosure in the form disclosed. Many modifications and variations in techniques and structures will be apparent to those of ordinary skill in an art without departing from a scope and spirit of this disclosure as set forth in the claims that follow. Accordingly, such modifications and variations are contemplated as being a part of this disclosure. A scope of this disclosure is defined by various claims, which include known equivalents and unforeseeable equivalents at a time of filing of this disclosure.

1. A device comprising:
at least two surfaces,
wherein one of the at least two surfaces is a face surface and the other of the at least two surfaces is a rear surface,
wherein the face surface is a moisture-wicking surface, wherein the rear surface is a moisture-absorbent surface, and
wherein the face surface faces a user and a rear surface is opposite to the face surface.

2. The device of claim 1, wherein the face surface comprises a hydrophobic yarn in an amount of about 29% to about 62% based on a total composition of the overall device weight.

3. The device of claim 1, wherein the amount of the hydrophobic yarn in the face surface is from about 49% to about 100%.

4. (canceled)

5. The device of claim 1, wherein the rear surface comprises a hydrophilic yarn in an amount of from about 38% to about 71% based on a total composition of the overall device weight.

6. (canceled)

7. (canceled)

8. The device of claim 1,
wherein the face surface comprises a hydrophobic yarn in an amount of about 29% to about 62% based on a total composition of a material of the face surface.

9. (canceled)

10. (canceled)

11. (canceled)

12. (canceled)

13. (canceled)

14. The device of claim 1;
wherein the rear surface comprises microfibers in an amount that is greater than an amount of microfibers in the face surface.

15. (canceled)

16. (canceled)

17. (canceled)

18. The device of claim 14, wherein the rear surface comprises a hydrophilic yarn in an amount of from about 38% to about 71%.

19. The device of claim 14, wherein the amount of the hydrophilic yarn in the rear surface is from about 51% to about 71%.

20. (canceled)

21. The device of claim 1, wherein the face surface comprises a moisture-wicking material selected from natural yarn, natural fibers, cellulosic fibers (seed, bast, leaf or grass), natural protein fibers (hair, fur, or secretion), synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof.

22. The device of claim 1, wherein the rear surface comprises a moisture absorbent material selected from natural yarn, natural fibers, cellulosic fibers (seed, bast, leaf or grass), natural protein fibers (hair, fur, or secretion), synthetic yarn, synthetic fibers (polyesters, polyamides, polyolefins, polyurethanes, elastomerics or acrylonitriles) manufactured cellulosic materials, recycled natural, recycled synthetics or other novel bio-based materials, or combinations thereof.

23. The device of claim 1,
wherein the at least two surfaces form a single layer, and
wherein a first surface of the single layer is the face surface and a second surface of the single layer opposite to the first layer is a rear surface.

24. (canceled)

25. The device of claim 1, further comprising a barrier layer positioned on the rear surface opposite to the face surface.

26. The device of claim 1, wherein the face surface comprises microfibers in an amount of 0.5-1.5 denier per filament.

27. The device of claim 1, wherein the face and rear surfaces are formed concurrently in a shaped knit pattern.

28. The device of claim 1, wherein each of the front and rear surfaces has a single or double pique pattern.

29. (canceled)

30. (canceled)

31. The device of claim 1, wherein the device has a moisture absorbance of about 5 ml to about 150 ml.

32. The device of claim 31, wherein the device has a moisture absorbance of about 25 ml to about 750 ml.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A method comprising:
causing a gusset performance structure to comprise at least two surfaces, wherein one of the at least two surfaces is a face surface and the other of the at least two surfaces is a rear surface,
causing the face surface to be positioned in a direction facing a user,
causing the rear surface to be positioned in a direction opposite to the face surface,
causing the face surface to comprise a moisture-wicking material, and
causing the rear surface to comprise a moisture-absorbent material.

47. The method of claim 46, further comprising forming the face and rear surfaces concurrently in a shaped knit pattern.

48. (canceled)

49. (canceled)

50. (canceled)

51. (canceled)

52. The method of claim 46, further comprising causing the face surface to comprise fibers or microfibers in an amount less than an amount of rear surface fibers or microfibers.
